(19) United States
(12) Patent Application Publication
(10) Pub. No.: US 2026/0144447 A1
(43) Pub. Date: May 28, 2026

(54) METHOD AND DEVICE FOR NONINVASIVELY MEASURING BLOOD PRESSURE, AND HOUSING

(71) Applicants: Wolfgang EGNER, Mainhausen (DE); Frank STROHMANN, Glattbach (DE)

(72) Inventors: Wolfgang EGNER, Mainhausen (DE); Frank STROHMANN, Glattbach (DE)

(21) Appl. No.: 19/104,234

(22) PCT Filed: Aug. 16, 2023
(86) PCT No.: PCT/DE2023/100605

§ 371 Date: Dec. 3, 2025

(30) Foreign Application Priority Data
Aug. 16, 2022 (DE) 10 2022 120 596.7

(51) Int. Cl. A61B 5/021; A61B 5/022

(52) U.S. Cl. CPC A61B 5/02125; A61B 5/022; A61B 2503/40

(57) ABSTRACT

The present invention relates to a method for noninvasively measuring blood pressure and to a device for noninvasively measuring blood pressure. The present invention also relates to a housing, especially for a device for noninvasively measuring blood pressure.

### CROSS REFERENCE TO RELATED APPLICATIONS

This application is a U.S. National Phase application under 35 U.S.C. § 371 of International Application No. PCT/DE2023/100605, filed on Aug. 16, 2023, and claims benefit to German Patent Application No. DE 10 2022 120 596.7, filed on Aug. 16, 2022. The International Application was published in German on Feb. 2, 2024 as WO 2024/037691 A1 under PCT Article 21 (2).

### FIELD

The present disclosure relates to a method for non-invasive blood pressure measurement and a device for non-invasive blood pressure measurement. Furthermore, the present disclosure relates to a housing, in particular for a device for non-invasive blood pressure measurement.

### BACKGROUND

Methods and devices for non-invasive blood pressure measurement are known from the state of the art. For example, a blood pressure measuring cuff is placed around an extremity of a living being, such as a human upper arm, and inflated with air. Air is then released from the cuff and the pressure curve in the cuff is measured. Based on the pressure values, the pressure changes in the cuff caused by the pulse waves of the living being can be determined and evaluated with regard to blood pressure parameters.

In the field of veterinary medicine in particular, however, the characteristics of the pulse waves and the typical blood pressure values can vary greatly depending on the respective animal species. It has therefore turned out to be technically and economically costly to determine the pulse waves and, based on this, the blood pressure parameters for different living beings both reliably and precisely and with simple and inexpensive means.

### SUMMARY

In an embodiment, the present disclosure provides a method that non-invasively measures blood pressure. The method includes: exerting a time-varying pressure on at least a part of a body of a living being using a compression unit at least during a measuring period. A first specific fluid pressure within a chamber of the compression unit is changed in a time-dependent manner at least during the measuring period by a first pressure adjustment unit. At different measuring times during the measuring period, at which at least partially different first specific fluid pressures are set, in each case at least one measured value of a differential pressure that exists at the respective measuring time between the fluid pressure within the chamber and the fluid pressure within a reference volume is recorded. A second specific fluid pressure within the reference volume is adjusted by a second pressure adjustment unit.

### BRIEF DESCRIPTION OF THE DRAWINGS

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 a schematic representation of a device according to an aspect of the present disclosure;

FIG. 2a a diagram of a course of the first and second specific fluid pressure in a chamber and a reference volume of a compression unit of the device of FIG. 1;

FIG. 2b a diagram of a course of the fluid pressure in the chamber of the compression unit of the device of FIG. 1;

FIG. 2c a diagram of a first course of pressure changes caused by pulse waves of a living being in the chamber of the compression unit of the device of FIG. 1 under first conditions;

FIG. 2d a diagram of a second course of pressure changes caused by pulse waves of a living being in the chamber of the compression unit of the device of FIG. 1 under second conditions;

FIG. 2e a diagram of a third course of pressure changes caused by pulse waves of a living being in the chamber of the compression unit of the device of FIG. 1 under third conditions;

FIG. 2f a diagram of a fourth course of pressure changes caused by pulse waves of a living being in the chamber of the compression unit of the device of FIG. 1 under fourth conditions;

FIG. 3 a flow chart of a method according to an aspect of the present disclosure;

FIG. 4a a schematic cross-sectional view of a housing according to an aspect of the present disclosure;

FIG. 4b an enlarged schematic detail representation of a section of FIG. 4a; and

FIG. 5 a schematic representation of parts of a device according to an aspect of the present disclosure.

### DETAILED DESCRIPTION

Aspects of the present disclosure overcome the described disadvantages of the state of the art and in particular to provide means by which non-invasive blood pressure measurement for different species of living beings, and in particular for different ranges of values of the blood pressure typical of living beings and of the pressure changes caused by the pulse waves, is possible both reliably and precisely and by simple and inexpensive means.

Aspects of the present disclosure provide a method for non-invasive blood pressure measurement, comprising exerting a time-varying pressure on at least a part of the body of a living being by means of a compression unit at least during a measuring period, in that a first specific fluid pressure within a chamber of the compression unit is changed in a time-dependent manner at least during the measuring period by means of a first pressure adjustment unit, wherein at different measuring times during the measuring period, at which at least partially different first specific fluid pressures are set, in each case at least one measured value of a differential pressure that exists at the respective measuring time between the, in particular current, fluid pressure within the chamber and the, in particular current, fluid pressure within a reference volume is recorded, and wherein a second specific fluid pressure within the reference volume is adjusted by means of a second pressure adjustment unit, in particular at least during the measuring period, wherein preferably by means of the second pressure adjustment unit the second specific fluid pressure within the reference volume (i) is adjusted at least during the measuring period and/or as a function of time and/or (ii) is adjusted in such a way, that at least at at least one, preferably at each, measuring time the, in particular current, fluid pressure within the reference volume is different from at least the first specific fluid pressure set at the respective measuring time and/or the fluid pressure existing in the chamber at the respective measuring time.

One or more aspects of the present disclosure are based on the surprising realization that a particularly reliable and accurate non-invasive blood pressure measurement, in particular including a pulse wave analysis, can be carried out if the available measuring range of the differential pressure sensor is used as fully as possible to detect the pressure changes caused by the pulse waves of the living being in the chamber of the compression unit.

By measuring the fluid pressure in the chamber against a fluid pressure in the reference volume, the required measuring range of the sensor can be reduced by adjusting the fluid pressure in the reference volume. At the same time, a larger proportion of the measuring range of the differential pressure sensor is available for detecting the pressure changes caused by the pulse waves. Due to the lower requirements on the measuring range, for example due to the reduced maximum pressure that the sensor used must be able to detect, cheaper differential pressure sensors can also be used. Higher resolutions can also be achieved for a sensor with a smaller measuring range at the same cost. This is because the bits of an analog/digital (AD) converter used become available for a reduced value range, which A/D converter, for example, converts an (analog) voltage signal supplied by the differential pressure sensor as a function of the pressure difference into a digital signal.

The person skilled in the art understands that during non-invasive blood pressure measurement, time-dependent pressure waves propagate through the blood vessel walls of the living being due to the heartbeat of the living being, thereby exerting time-dependent forces on the compression unit and thus on the chamber. These forces lead to a time-dependent change in volume in the chamber and thus contribute to a time-dependent change in the fluid pressure of the fluid in the chamber. The acting forces, and thus the pressure conditions within the chamber, can be, and advantageously are, dependent on the pressure with which the compression unit acts on the part of the body of the living being. Based on the course of the pressure changes caused by the pulse waves within the chamber for different external pressures of the compression unit, blood pressure parameters can advantageously be determined in an at least partially known manner.

For example, when performing a conventional oscillatory measurement in a healthy human, the pulse waves typically cause a pressure difference of 2.5 mmHg at the peak and between 0.9 and 2.5 mmHg over the entire evaluation, and between 0.22 and 0.9 μHg in a healthy cat, in each case in the chamber of the compression unit when typical fluid pressures are built up in the chamber. These fluid pressures (i.e. the first specific fluid pressure in each case) generally range from above 130 mmHg to below 80 mmHg in human measurements and from 300 mmHg to 10 mmHg in a cat, for example.

By measuring the fluid pressure in the chamber not against atmosphere but against the fluid pressure in the reference volume as proposed, the measuring range of the sensor (such as a differential pressure sensor preferably used for this purpose) does not have to be designed for the maximum pressures occurring in the chamber. Preferably, the necessary measuring range is determined by the difference between the first and second specific fluid pressure and by the typical pressure changes in the chamber due to the pulse waves. In this way, the range of values that can be detected by the sensor can be reduced to 50 mmHg (in humans) and 10 mmHg (in a cat), for example.

At the same time, the bits (around 8 bits) of the A/D converter used are then available for a smaller value range. So if the value range to be recorded with the pressure sensor can be reduced from 0 . . . 150 mmHg to 0 . . . 50 mmHg, the resolution is improved from the previous 0.586 mmHg/bit (150 mmHg/256 bits=0.586 mmHg/bit) to 0.195/bit (50 mmHg/256 bits=0.195 mmHg/bit) with the same 8-bit A/D converter.

This means that sensors with a significantly smaller measuring range can be used and still provide even more information. This also saves costs, as sensors with a smaller measuring range are often cheaper. This is because the sensor only has to be designed for significantly smaller amplitude values. At the same time, reducing the measuring range makes it possible to increase the resolution at the same or even lower cost.

The number of bits can also be reduced while maintaining the same resolution, for example, which makes it possible to increase the sampling rates, i.e. the recording rates of successive measured values.

The disclosed method makes it possible to record pulse waves from living organisms, which cause pressure changes in the chamber in the range of only sub-mmHg, as is typically the case with many living organisms, such as cats (pressure changes here are in the range of only μHg, for example), with significantly less economic effort and even with improved accuracy.

The disclosed method makes it possible to change the, at least imaginary, baseline of the pulse waves (compared to a pressure measurement against atmosphere), in particular to shift and/or tilt it. This also makes the method very flexible.

In this context, the inventors recognized above all that the disclosed method makes it very easy to align the course of the pressure fluctuations caused by the pulse waves in such a way that the pressure values of the pulse waves are oriented around the zero line, preferably symmetrically. This means that the course of the pulse waves covers both positive and negative pressure values. This means that an A/D converter can be used that is also designed for negative values. This means that this value range can be utilized particularly well and is not lost. The method therefore enables the flexible use of different A/D converters.

In addition, the method advantageously enables the pulse waves to be recorded in a course mirrored on a line, in particular a horizontal line. For this purpose, it is sufficient, and is also realized in a preferred embodiment, to perform the differential value formation in such a way that the fluid pressure in the chamber is subtracted from the fluid pressure in the reference volume. This can be done particularly easily by connecting the two sides of a differential pressure sensor to the chamber and the reference volume.

Preferably, the positioning of the baseline and/or orientation (in particular in the form of a mirror image) of the pulse waves can be influenced by the choice of the second specific fluid pressure in the reference volume, by choosing whether the second specific fluid pressure in the reference volume is greater or less than the first specific fluid pressure in the chamber and/or by selecting how the difference between the fluid pressures in the chamber and reference volume is formed, i.e. whether the fluid pressure in the reference volume is subtracted from the fluid pressure in the chamber or whether the fluid pressure in the chamber is subtracted from the fluid pressure in the reference volume.

In addition, the inventors found that undefined pressure values were recorded as a result of disturbing influences, which could be caused by actuation of the valves and/or pumps used to adjust the first specific fluid pressure in the chamber or by temporary muscle relaxation or twitching of the living being and a resulting change in pressure in the chamber. By being able to adjust the baseline in the present case, it can be advantageously ensured that the differential pressure sensor and/or the A/D converter is or are operated within a permissible value range. This means that artifacts can be reliably detected and evaluated when recording measured values.

Preferably, the fluid pressure in the chamber (in particular at least during the measuring period) is therefore determined by several influences. The fluid pressure in the chamber is advantageously determined at least by the time-dependent first specific fluid pressure and a time-dependent volume change within the chamber caused by the pulse waves. The time-dependent volume change advantageously represents the pulse waves (which propagate via the blood vessel walls with each heartbeat), which can therefore be measured as pressure variations and can be represented at least mentally as superimposed on the first specific fluid pressure.

The fluid pressure in the chamber can therefore advantageously be represented as a composition (in particular as an additive superposition) of the first specific fluid pressure and the pressure changes due to the pulse waves. In other words, the time-dependent pressure changes due to the pulse waves are imprinted on the course of the first specific fluid pressure. In fact, the course of the first specific fluid pressure can advantageously be taken from the course of the fluid pressure in the chamber (in particular measured at least during the measuring period and/or against atmosphere) as a (for example falling) “baseline” of the pulse waves.

The fluid pressure in the chamber at a certain point in time (i.e. the current fluid pressure) is then advantageously influenced by at least the first specific fluid pressure and the change in volume of the chamber as a result of the influence of the pulse waves at this point in time.

Preferably, the contribution to the fluid pressure within the chamber that can be changed by means of the first pressure adjustment unit is referred to as the first specific fluid pressure.

Preferably, the contribution to the fluid pressure within the reference volume that can be changed by means of the second pressure adjustment unit is referred to as the second specific fluid pressure.

In one embodiment, the method for non-invasive blood pressure measurement may be or comprise a method for pulse wave analysis and/or pulse wave recording.

In preferred embodiments, the time-varying pressure exerted on the body part during the measuring period can change continuously. However, it may also be preferred that the time-varying pressure exerted on the body part during the measuring period is at least temporarily constant. This makes it advantageously possible to record measured values at a constant applied pressure. Thus, for example, the pulse waves can be recorded and/or analyzed at a constant applied pressure.

In the present disclosure, the respective pressures are indicated in the unit mmHg (millimeters of mercury), which is commonly used in the field of blood pressure measurement. Where reference is made to very low pressure values, the unit μHg (1 mmHg=1000 μHg) is also used in some cases.

It is clear to the skilled person that the (single) pulse wave propagating from the heart via the blood vessels can differ depending on the location in the body and is also influenced, for example, at the measurement location by the pressure exerted on the body part by the compression unit. In the present disclosure, however, the measured course of the pressure changes caused by the (several successive) pulse waves is itself sometimes also referred to as a pulse wave. However, the skilled person understands from the respective context whether the actual (single or several successive) pulse wave(s) emanating from the heart is or are meant, or whether the course of the pressure differences occurring in the chamber over several heartbeats is meant. Pulse waves recorded over several heartbeats typically have several (local) amplitudes, which were preferably recorded at least partially with different pressures acting on the body part, and the level of which can be used to determine blood pressure parameters, for example.

In the present disclosure, the width of a (pressure) value range is preferably understood to be the difference between the maximum and minimum (pressure) value of this (pressure) value range, unless something to the contrary arises from the respective context at the relevant point. The (pressure) value range 80 mmHg to 130 mmHg therefore has a width of 50 mmHg (130 mmHg-80 mmHg).

Preferably, in the present disclosure, the differential pressure ΔP between a first pressure P₁ and a second pressure P₂ is measured as ΔP=P₁−P₂, unless something to the contrary arises from the respective context. So if the first pressure is greater than the second pressure, the differential pressure ΔP assumes positive values. And if the second pressure is therefore greater than the first pressure, the differential pressure ΔP assumes negative values. Optionally, in embodiments, the differential pressure can also be defined as ΔP=P₂−P₁, in which case the opposite signs apply.

Preferably, the chamber and the reference volume are fluidically separated from each other, especially at least during the measuring period. This is a particularly advantageous way of ensuring that the fluid pressure in the reference volume is not influenced by the fluid pressure in the chamber. In this way, the fluid pressure in the reference volume can be used particularly reliably as a reference value and the pulse waves can be recorded very accurately.

The measuring period can be defined or definable. For example, the measuring period is more than 10 seconds, less than 600 seconds and/or between 10 seconds and 600 seconds. Preferably, the measuring period is 30 seconds or more, 60 seconds or more and/or 120 seconds or more. Particularly preferred measuring periods are between 10 and 30 seconds or between 30 and 60 seconds. Alternatively or additionally, the measuring period can advantageously be defined by a period within which the differential pressure measurements relevant for the blood pressure measurement lie.

In one embodiment, the measuring period is selected at least partially depending on the pulse rate of the living being. For this purpose, the pulse rate of the living being is optionally determined.

The differential pressure measured at a measuring time is advantageously the pressure difference between the current fluid pressures in the chamber and the reference volume at the respective measuring time.

Preferably, the chamber is provided within the compression unit.

Preferably, the compression unit and/or the chamber can be deformed at least in certain areas and/or is designed in such a way that the volume within the chamber (and thus the fluid pressure in the chamber) can be changed by applying force (in particular due to the pulse waves) to the compression unit. For example, this can be realized particularly advantageously if the compression unit is a blood pressure measuring cuff. Therefore, the compression unit is advantageously a blood pressure measuring cuff or has one. The chamber can then be provided in the cuff and have a volume enclosed within the cuff.

Preferably, the chamber has a volume within which the fluid pressure exists and/or within which the first specific fluid pressure is adjustable. The chamber may be provided within a blood pressure measuring cuff. In this case, the chamber and the volume can be provided with the cuff in a manner known per se

Preferably, the first pressure adjustment unit has a first pressure sensor. This first pressure sensor preferably measures the respective fluid pressure in the chamber against atmosphere. In one embodiment, the first pressure sensor is used to monitor and/or control the fluid pressure, in particular the first specific fluid pressure, in the chamber.

Preferably, the second pressure adjustment unit has a second pressure sensor. This second pressure sensor preferably measures the respective fluid pressure in the reference volume against the atmosphere. In one embodiment, the second pressure sensor is used to monitor and/or control the fluid pressure, in particular the second specific fluid pressure, in the reference volume.

In one embodiment, the setting of the pressure difference between the first and second specific fluid pressure, so that at least at each measuring time the fluid pressure within the reference volume is different from at least the first specific fluid pressure set at the respective measuring time, comprises that values of the first pressure sensor, the second pressure sensor and/or the differential pressure sensor are included, in particular are processed as part of a control and/or regulation system.

In one embodiment, a computing unit can be provided which is in operative connection with one or both pressure adjustment units in order to be able to influence, monitor, control and/or regulate them in a suitable manner. In this way, the first specific fluid pressure in the chamber and/or the second specific fluid pressure in the reference volume can be reliably adjusted (and as described above).

The fluid in the chamber and/or in the reference volume can advantageously be air.

The measuring period can advantageously be defined or definable in terms of its length.

For example, the measured values can be recorded at a rate of at least 10 per second, at most 10,000 per second and/or between 10 per second and 10,000 per second. Preferred recording rates are between 10 and 100 per second, 100 and 500 per second, 300 and 800 per second, 500 and 1000 per second and/or 800 and 1500 per second. Recording rates of 10 per second, 100 per second, 500 per second and/or 1000 per second are particularly preferred.

As mentioned above, the proposed method can also be used to achieve comparatively high recording rates, making it possible to carry out meaningful blood pressure tests.

In one embodiment, the recording rate of the measured values is selected at least partially as a function of the pulse rate of the living being. For this purpose, the pulse rate of the living being is optionally determined. This can advantageously ensure that every pulse wave emanating from the heart is detected.

In one embodiment, the individual measured values are recorded at equal time intervals. The inverse recording rate then represents the time interval between two measured values.

The living being can be a human or an animal, such as a mammal, for example a dog, cat, hamster, mouse, horse, hare, rabbit or rat, bird, reptile or amphibian.

The part of the body of the living being can, for example, have blood flowing through it and/or be an extremity of the living being, in particular one with blood flowing through it, such as an upper arm of a human or a tail of an animal.

In one embodiment, the second specific fluid pressure within the reference volume is adjusted by means of the second pressure adjustment unit in such a way that the fluid pressure, in particular the current fluid pressure, within the reference volume is also different from the ambient pressure at least at each measuring time.

The individual components for non-invasive blood pressure measurement, i.e. in particular compression unit, first pressure adjustment unit, second pressure adjustment unit and/or means for recording the differential pressure (such as a differential pressure sensor), can advantageously be comprised by a device for non-invasive blood pressure measurement and/or can be provided individually and/or together for the method.

Alternatively or additionally, it may also be provided that the first specific fluid pressure is set within the chamber, in particular by means of the first pressure adjustment unit and/or before the measuring period, to a first initial pressure value, which is preferably above the systolic blood pressure of the living being, and preferably the first specific fluid pressure is changed in a time-dependent manner starting from the first initial pressure value, in particular at least during the measuring period, preferably until a first target pressure value, which is preferably below the diastolic blood pressure of the living being, is reached.

This means that the measurement for a non-invasive blood pressure measurement can be carried out in a very defined manner and under known conditions. In particular, this also favors repeatability, so that different measurements can be compared with each other in a particularly advantageous way. As a result, deviations between two measurements, which may indicate a disease in the living being, can be reliably detected.

The first initial pressure value can be defined or definable. The first target pressure value can be defined or definable.

Preferably, the first specific fluid pressure is changed from the first initial pressure value as a function of time from a point in time that is before the start of the measuring period or that represents the start of the measuring period.

Preferably, the first specific fluid pressure reaches the first target pressure value at a point in time that is after the end of the measuring period or that represents the end of the measuring period.

For example, the first initial pressure value can be based on the systolic blood pressure and be above this value and/or the first target pressure value can be based on the diastolic blood pressure and be below this value.

For example, the first initial pressure value can be based on the diastolic blood pressure and lie below this value and/or the first target pressure value can be based on the systolic blood pressure and lie above this value.

For example, the chamber can first be inflated until the first specific fluid pressure has reached the first initial pressure value (which can take into account the above-mentioned exemplary values) and then the first specific fluid pressure can be changed until the defined or definable first target pressure value (which can take into account the above-mentioned exemplary values) is reached.

For example, it may be provided that while the first specific fluid pressure is brought to the first initial pressure value, the fluid pressure, in particular the first specific fluid pressure, in the chamber is monitored by means of the first pressure sensor and/or the first specific fluid pressure is controlled.

For example, it may be provided that while the first specific fluid pressure is changed to the first target pressure value, the fluid pressure, in particular the first specific fluid pressure, in the chamber is monitored by means of the first pressure sensor and/or the first specific fluid pressure is controlled.

In a preferred embodiment, the first specific fluid pressure set at the first measuring time during the measuring period is smaller or higher than the first initial pressure value. In other words, the measuring period can be timed to start after the first specific fluid pressure has already been changed starting from the first initial pressure value. This is therefore particularly preferable, since during the period up to the first measuring time, any control electronics used can thus adjust itself advantageously. Similarly, the first specific fluid pressure set at the last measuring time during the measuring period can alternatively or additionally be greater or less than the first target pressure value.

Alternatively or additionally, it may also be provided that (A) the first initial pressure value (i) is greater than 10 mmHg, preferably greater than 50 mmHg, preferably greater than 100 mmHg, preferably greater than 150 mmHg, preferably greater than 200 mmHg, preferably greater than 250 mmHg, preferably greater than 300 mmHg, preferably greater than 350 mmHg, preferably greater than 400 mmHg, preferably greater than 450 mmHg, preferably greater than 500 mmHg, (ii) less than 700 mmHg, preferably less than 600 mmHg, preferably less than 550 mmHg, preferably less than 500 mmHg, preferably less than 450 mmHg, preferably less than 400 mmHg, preferably less than 350 mmHg, preferably less than 300 mmHg, preferably less than 250 mmHg, preferably less than 200 mmHg, preferably less than 150 mmHg, preferably less than 100 mmHg, preferably less than 50 mmHg, and/or (iii) between 10 mmHg and 700 mmHg, preferably between 100 mmHg and 500 mmHg, preferably between 100 mmHg and 400 mmHg, preferably between 150 mmHg and 350 mmHg, preferably between 150 mmHg and 200 mmHg or between 200 mmHg and 350 mmHg, and/or

(B) the first target pressure value (i) greater than 1 mmHg, preferably greater than 3 mmHg, preferably greater than 5 mmHg, preferably greater than 10 mmHg, preferably greater than 20 mmHg, preferably greater than 30 mmHg, preferably greater than 40 mmHg, preferably greater than 50 mmHg, preferably greater than 60 mmHg, preferably greater than 70 mmHg, preferably greater than 100 mmHg, (ii) less than 300 mmHg, preferably less than 250 mmHg, preferably less than 200 mmHg, preferably less than 150 mmHg, preferably less than 100 mmHg, preferably less than 80 mmHg, preferably less than 50 mmHg, preferably less than 30 mmHg, preferably less than 20 mmHg, preferably less than 15 mmHg, preferably less than 10 mmHg, preferably less than 5 mmHg, preferably less than 3 mmHg, and/or (iii) between 1 mmHg and 300 mmHg, preferably between 1 mmHg and 200 mmHg, preferably between 1 mmHg and 100 mmHg, preferably between 1 mmHg and 80 mmHg, preferably between 1 mmHg and 20 mmHg or between 20 mmHg and 80 mmHg.

Alternatively or additionally, it may also be provided that the first specific fluid pressure is changed continuously or stepwise, in particular at least during the measuring period, by means of the first pressure adjustment unit.

A continuous change in the first specific fluid pressure can be achieved, for example, by continuously removing the fluid from the chamber or pumping it into the chamber.

Preferably, a stepwise change in the first specific fluid pressure is understood to mean a course of the first specific fluid pressure which repeatedly, in particular periodically, remains at least temporarily at a constant pressure level and wherein between two immediately successive pressure levels, a first and an immediately following second pressure level, the first specific fluid pressure changes from the first pressure level to the second pressure level, in particular at least temporarily in a straight line and/or at least temporarily according to a curved course.

Alternatively or additionally, it may also be provided that the first specific fluid pressure, in particular at least during the measuring period, changes at a rate, in particular a continuous rate, of (i) 1 mmHg or more than 1 mmHg per second, (ii) 50 mmHg or less than 50 mmHg per second, preferably 30 mmHg or less than 30 mmHg per second, preferably 20 mmHg or less than 20 mmHg per second, and/or (iii) between 1 mmHg and 50 mmHg per second, preferably between than 3 mmHg per second and 30 mmHg per second.

Preferred rates of change are 1 mmHg per second, 3 mmHg per second, 5 mmHg per second, 10 mmHg per second, 15 mmHg per second, 20 mmHg per second, 25 mmHg per second and/or 27 mmHg per second.

If the living being is a human, a rate of change of between 3 mmHg and 27 mmHg per second is particularly preferred.

In one embodiment, the rate of change is selected at least partially as a function of the pulse rate of the living organism. For this purpose, the pulse rate of the living being is optionally determined. This can advantageously ensure that every pulse wave emanating from the heart is detected.

Alternatively or additionally, it may also be provided that the first specific fluid pressure is changed, in particular at least during the measuring period, with a stepwise rate of change of (i) 1 mmHg or more than 1 mmHg, (ii) 50 mmHg or less than 50 mmHg, and/or (iii) between 1 mmHg and 50 mmHg.

The rate of change is preferably to be regarded as the pressure difference between two of the immediately successive pressure levels described above.

Preferred rates of change are 1 mmHg, 3 mmHg, 5 mmHg, 10 mmHg, 15 mmHg, 20 mmHg, 25 mmHg and/or 27 mmHg.

Alternatively or additionally, it may also be provided that the time-dependent variation of the first specific fluid pressure comprises or represents the time-dependent reduction of the first specific fluid pressure.

For example, the first specific fluid pressure can be reduced from the first initial pressure value to the first target pressure value.

Alternatively or additionally, it may also be provided that the time-dependent change in the first specific fluid pressure comprises or represents the time-dependent increase in the first specific fluid pressure.

For example, the first specific fluid pressure can be increased from the first initial pressure value to the first target pressure value.

Alternatively or additionally, it may also be provided that, in particular at least during the measuring period, the fluid pressure, in particular the current one, existing in the chamber at least at individual measuring times results at least from the time-dependent first specific fluid pressure and a time-dependent action pressure exerted on the chamber by the part of the body of the living being, wherein the action pressure is preferably influenced at least partially and/or at least temporarily by the blood pressure of the living being and/or by pulse waves propagating via the blood vessels of the living being.

In other words, the fluid pressure that can be measured in the chamber (i.e. the current fluid pressure) is influenced by several factors.

One factor of at least two factors is preferably the pressure increase caused by the first pressure adjustment unit in accordance with the first specific fluid pressure compared to atmosphere. Another factor of the at least two factors is preferably the forces ultimately acting on the compression unit and thus on the chamber as a result of the pulse beat of the living being mediated by the pulse waves, which lead to a change in volume in the chamber and thus to a change in the fluid pressure of the fluid in the chamber in accordance with the course of the pulse waves.

Of course, it is possible that the factors cannot be separated from each other, at least on the basis of a single measured value of the fluid pressure in the chamber compared to the atmosphere. However, for example, especially if the course of the fluid pressure in the chamber were to be recorded over time, the pulse waves could run along an (imaginary) curve of a pressure curve according to the first specific fluid pressure.

Alternatively or additionally, it may also be provided that the second specific fluid pressure within the reference volume is set to a second initial pressure value, in particular by means of the second pressure adjustment unit and/or before the measuring period, and/or is changed as a function of time, in particular by means of the second pressure adjustment unit, starting from the second initial pressure value and/or at least during the measuring period, preferably until a second target pressure value is reached.

Preferably, the second specific fluid pressure is changed from the second initial pressure value as a function of time from a point in time that is before the start of the measuring period or that represents the start of the measuring period.

Preferably, the second specific fluid pressure reaches the second target pressure value at a point in time that is after the end of the measuring period or that represents the end of the measuring period.

The second initial pressure value can be defined or definable. The second target pressure value can be defined or definable.

For example, the reference volume can first be inflated until the second specific fluid pressure has reached the second initial pressure value and then the second specific fluid pressure can be changed until the defined or definable second target pressure value is reached.

For example, it may be provided that while the second specific fluid pressure is brought to the second initial pressure value, the fluid pressure, in particular the second specific fluid pressure, in the reference volume is monitored by means of the second pressure sensor and/or the second specific fluid pressure is controlled.

For example, it may be provided that while the second specific fluid pressure is changed to the second target pressure value, the fluid pressure, in particular the second specific fluid pressure, in the reference volume is monitored by means of the second pressure sensor and/or the second specific fluid pressure is controlled.

In a preferred embodiment, the second specific fluid pressure set at the first measuring time during the measuring period is smaller or higher than the second initial pressure value. In other words, the measuring period can be timed to start after the second specific fluid pressure has already been changed starting from the second initial pressure value. This is therefore particularly preferable, since during the period up to the first measuring time, any control electronics used can thus adjust itself advantageously. Similarly, the second specific fluid pressure set at the last measuring time during the measuring period can alternatively or additionally be greater or less than the second target pressure value.

In an also preferred embodiment, the following may be provided: (i) the first specific fluid pressure, in particular while the fluid pressure, in particular the first specific fluid pressure, in the chamber is monitored and/or controlled by means of the first pressure sensor, is brought to the first initial pressure value or a value deviating therefrom by at most 50%, preferably by at most 40%, preferably by at most 30%, preferably by at most 20%, preferably by at most 10%, preferably by at most 5%, preferably by at most 3%, preferably by at most 1%, and/or the second specific fluid pressure, in particular while the fluid pressure, in particular the second specific fluid pressure, in the reference volume is monitored and/or controlled by means of the second pressure sensor, is brought to the second initial pressure value or to a value deviating therefrom by at most 50%, preferably by at most 40%, preferably by at most 30%, preferably by at most 20%, preferably by at most 10%, preferably by at most 5%, preferably by at most 3%, preferably by at most 1%, and/or (ii) a defined or definable pressure difference between the fluid pressure within the chamber and the fluid pressure within the reference volume is set, in particular subsequently and/or during this process, by means of a control system taking into account measured values from the differential pressure sensor.

A fluid pressure preferably deviates by a maximum of X % from an initial pressure value if the ratio of “fluid pressure in mmHg” and “initial pressure value in mmHg” is between 1−X/100 and 1+X/100.

In other words, one of the two fluid pressures within the chamber and the reference volume or both fluid pressures can be brought to an approximate value. A low-cost pressure sensor can be used for this purpose, as a comparatively low resolution is sufficient. The pressure difference between the chamber and the reference volume can then be set to the desired value using the much finer resolution differential pressure sensor.

Alternatively or additionally, it may also be provided that (A) the second initial pressure value (i) is greater than 10 mmHg, preferably greater than 50 mmHg, preferably greater than 100 mmHg, preferably greater than 150 mmHg, preferably greater than 200 mmHg, preferably greater than 250 mmHg, preferably greater than 300 mmHg, preferably greater than 350 mmHg, preferably greater than 400 mmHg, preferably greater than 450 mmHg, preferably greater than 500 mmHg, (ii) is less than 700 mmHg, preferably less than 600 mmHg, preferably less than 550 mmHg, preferably less than 500 mmHg, preferably less than 450 mmHg, preferably less than 400 mmHg, preferably less than 350 mmHg, preferably less than 300 mmHg, preferably less than 250 mmHg, preferably less than 200 mmHg, preferably less than 150 mmHg, preferably less than 100 mmHg, preferably less than 50 mmHg, and/or (iii) is between 10 mmHg and 700 mmHg, preferably between 100 mmHg and 500 mmHg, preferably between 100 mmHg and 400 mmHg, preferably between 150 mmHg and 350 mmHg, preferably between 150 mmHg and 200 mmHg or between 200 mmHg and 350 mmHg,
and/or
(B) the second target pressure value (i) is greater than 1 mmHg, preferably greater than 3 mmHg, preferably greater than 5 mmHg, preferably greater than 10 mmHg, preferably greater than 20 mmHg, preferably greater than 30 mmHg, preferably greater than 40 mmHg, preferably greater than 50 mmHg, preferably greater than 60 mmHg, preferably greater than 70 mmHg, preferably greater than 100 mmHg, (ii) is less than 300 mmHg, preferably less than 250 mmHg, preferably less than 200 mmHg, preferably less than 150 mmHg, preferably less than 100 mmHg, preferably less than 80 mmHg, preferably less than 50 mmHg, preferably less than 30 mmHg, preferably less than 20 mmHg, preferably less than 15 mmHg, preferably less than 10 mmHg, preferably less than 5 mmHg, preferably less than 3 mmHg, and/or (iii) is between 1 mmHg and 300 mmHg, preferably between 1 mmHg and 200 mmHg, preferably between 1 mmHg and 100 mmHg, preferably between 1 mmHg and 80 mmHg, preferably between 1 mmHg and 20 mmHg or between 20 mmHg and 80 mmHg.

This allows the fluid pressure within the reference volume to be set very reliably and/or changed over time.

Alternatively or additionally, it may also be provided that the second specific fluid pressure, in particular at least during the measuring period, (i) is adjusted within the reference volume in such a way that the ratio of the first specific fluid pressure in the chamber and the second specific fluid pressure within the reference volume, in particular at each measuring time point, has a value (a) of between 0.5 and less than 1.0, in particular of between 0.6 and less than 1.0, in particular of between 0.7 and less than 1.0, in particular of between 0.9 and less than 1.0, and/or (b) of between more than 1.0 and 1.5, in particular of between more than 1.0 and 1.4, in particular of between more than 1.0 and 1.3, in particular of between more than 1.0 and 1.1, (ii) is changed within the reference volume at least temporarily simultaneously with the first specific fluid pressure within the chamber, (iii) is changed within the reference volume in phase with the first specific fluid pressure within the chamber, (iv) has within the reference volume a pressure difference in terms of magnitude with respect to the first specific fluid pressure within the chamber of at least one times, preferably at least two times, preferably at least three times, preferably at least four times, preferably at least five times, the maximum pressure change caused by the pulse waves during the measuring period and/or (v) is changed within the reference volume at a rate of change identical to that of the first specific fluid pressure within the chamber.

The two fluid pressures are preferably changed in phase if an increase in the fluid pressure in the chamber is matched by an increase in the fluid pressure in the reference volume or vice versa, and if a decrease in the fluid pressure in the chamber is matched by a decrease in the fluid pressure in the reference volume or vice versa. The fluid pressure can be changed in the chamber and reference volume simultaneously or with a time delay in the same phase, in particular by adjusting the first specific fluid pressure and/or the second specific fluid pressure.

Preferably, an identical rate of change of the fluid pressures is present if the two rates of change differ from each other by at most 10%, preferably at most 7%, preferably at most 5%, preferably at most 3%, preferably at most 2%, preferably at most 1%, preferably at most 0.5%.

Alternatively or additionally, it may also be provided that, in particular at least at the individual measuring times, the pressure difference between the first specific fluid pressure within the chamber and the second specific fluid pressure within the reference volume is set (i) to a constant value and/or (ii) to a value of (a) 1 mmHg or more than 1 mmHg, (b) 600 mmHg or less than 600 mmHg and/or (c) between 1 mmHg and 600, preferably between 1 mmHg and 100 mmHg, preferably between 10 mmHg and 50 mmHg, such as 1 mmHg, 10 mmHg, 25 mmHg, 30 mmHg or 50 mmHg.

Advantageous pressure differences have values of 25 mmHg, 50 mmHg or 100 mmHg, for example.

A constant pressure difference here therefore preferably means that there is the same pressure difference between the first specific fluid pressure in the chamber and the second specific fluid pressure in the reference volume, in particular at each measuring time. Either the first specific fluid pressure in the chamber can in each case be greater than the second specific fluid pressure in the reference volume or, conversely, the first specific fluid pressure in the chamber can in each case be less than the second specific fluid pressure in the reference volume.

In one embodiment, the setting of the constant pressure difference has the effect that values from the first pressure sensor, the second pressure sensor and/or the differential pressure sensor are included, in particular are processed as part of a control and/or regulation system.

Alternatively or additionally, it may also be provided that the fluid pressure, in particular the current fluid pressure, within the reference volume is not influenced by the living being or parts thereof, in particular not by the blood pressure of the living being and/or by pulse waves propagating via the blood vessels of the living being.

Preferably, therefore, the reference volume is provided and/or made available in such a way that it is not in operative connection with the living being, in particular is not arranged on the living being and/or is in contact with it. For example, the reference volume can be provided separately and/or spatially separated from the living organism.

In one embodiment, the fluid pressure in the reference volume, in particular for given ambient conditions, such as in particular a given atmospheric pressure, is determined only by the second specific fluid pressure.

Alternatively or additionally, it may also be provided that the first specific fluid pressure in the chamber and the second specific fluid pressure in the reference volume are controlled and/or adjusted, in particular at least during the measuring period and/or at least for each measurement, in such a way that, in particular at least during the measuring period and/or at least for each measurement, the first specific fluid pressure is, preferably at any time, at a greater or smaller value than the second specific fluid pressure.

In one embodiment, the first specific fluid pressure within the chamber and the second specific fluid pressure within the reference volume are each set to the first or second initial pressure value, which, however, differ, and starting from the respective initial pressure value, the first specific fluid pressure within the chamber and the second specific fluid pressure within the reference volume are changed simultaneously and/or at an identical rate of change, for example reduced or increased, wherein, for example, the pressure difference between the first specific fluid pressure and the second specific fluid pressure is always kept constant.

Alternatively or additionally, it can also be provided that the recorded measured values result in a measurement curve which represents and/or is and/or makes it possible to determine a course of the pulse waves of the blood pressure of the living being.

For example, a curve can be laid through the measured values using the method of least squares in order to obtain a measurement curve and/or the course of the pulse waves.

Alternatively or additionally, it may also be provided that the recorded measured values are evaluated and/or blood pressure parameters, such as systolic blood pressure, diastolic blood pressure and/or morphological characteristics of the living being are determined based on at least one result of the evaluation.

In one embodiment, the evaluation of the measured values comprises determining and evaluating an envelope for the measured values and determining the blood pressure parameters based on at least one result of the evaluation of the envelope.

The envelope can be evaluated in a known manner. For example, the envelope of measured values recorded during a decreasing first specific fluid pressure can increase at a first first specific fluid pressure (here the distinction is made between presystolic pulse pressure amplitude with the artery closed and the first opening of the artery), wherein preferably this first first specific fluid pressure then represents the systolic blood pressure of the living being, can reach its maximum at a second first specific fluid pressure, wherein preferably this second first specific fluid pressure then represents the mean arterial pressure (MAP) of the living being, and can fall again at a third first specific fluid pressure, wherein preferably this third first specific fluid pressure then represents the diastolic blood pressure of the living being.

In this way, the above-mentioned blood pressure parameters can be determined in a particularly advantageous way using the recorded measured values and/or the envelopes.

Preferably, the amplitudes of the individual pulse waves are evaluated and preferably compared with each other. This is particularly advantageous for recognizing morphological changes in the amplitudes and pulse waves.

In one embodiment, the recorded measured values, in particular the course of the pulse waves, the blood pressure parameters and/or at least one result of the evaluation are output graphically, for example on a display device.

Morphological characteristics of the creature may include one or more of the following options: Pulse pressure, amplitude volume, pulse spacing beat to beat, amplitude width and height, and/or respiration.

In one embodiment, the measured values are evaluated with the calculation unit as described above.

Alternatively or additionally, it may also be provided that the differential pressure between the, in particular current, fluid pressure in the chamber and the, in particular current, fluid pressure in the reference volume is measured by means of a differential pressure sensor, wherein preferably a first side of the differential pressure sensor is fluidly connected to the chamber and a second side of the differential pressure sensor is fluidly connected to the reference volume, and in particular the differential pressure sensor supplies positive pressure values, if the, in particular current, fluid pressure in the chamber is greater than the, in particular current, fluid pressure in the reference volume, and the differential pressure sensor supplies negative pressure values if the, in particular current, fluid pressure in the chamber is less than the, in particular current, fluid pressure in the reference volume.

The first side of the differential pressure sensor is therefore advantageously subjected to the fluid pressure within the chamber and/or the second side of the differential pressure sensor is subjected to the fluid pressure within the reference volume.

Preferably, the differential pressure sensor has a measuring range that corresponds to at least one, preferably at least two, preferably at least three, preferably at least four, preferably at least five times the expected width of the value range of the pulse waves of the living organism.

For example, the differential pressure sensor can have a measuring range of at least 10 mmHg or at least 30 mmHg or at least 50 mmHg.

Advantageously, it may be provided that the computing unit is operatively connected to the differential pressure sensor and/or that the computing unit receives the measured values from the differential pressure sensor.

Alternatively or additionally, it may also be provided that the first pressure adjustment unit comprises a first electronic valve and/or a first pump, such as a piezo pump, and/or wherein the time-dependent changing of the first specific fluid pressure comprises controlling and/or regulating the first electronic valve and/or the first pump in a time-dependent manner.

A corresponding valve and/or pump can be regulated and/or controlled particularly reliably and by inexpensive means.

Alternatively or additionally, it may also be provided that the second pressure adjustment unit comprises a second electronic valve and/or a second pump, such as a piezo pump, and/or wherein the time-dependent changing of the second specific fluid pressure within the reference volume comprises controlling and/or regulating the second electronic valve and/or the second pump in a time-dependent manner.

A corresponding valve and/or pump can be regulated and/or controlled particularly reliably and by inexpensive means.

Alternatively or additionally, it may also be provided that the compression unit comprises or is a blood pressure measuring cuff, wherein preferably the part of the body of the living being can be compressed to varying degrees by the cuff, and/or wherein the chamber is a chamber of the cuff, which preferably can be filled with a fluid and emptied.

In order to carry out a non-invasive blood pressure measurement, the method can therefore advantageously provide that the compression unit, in particular the blood pressure measuring cuff, is first arranged on the living being, for example by placing it around the extremity of the living being. Preferably, the first specific fluid pressure in the chamber is then increased, for example by inflating the chamber with fluid (such as air) (for example by means of the first pump). This is preferably done until the first specific fluid pressure reaches the first initial pressure value.

Alternatively or additionally, it may also be provided that the reference volume is a volume (i) within an object, (ii) within a hollow body and/or (iii) within a container.

It has been found to be particularly advantageous that the size of the reference volume does not have to be based on the size of the volume of the chamber, as only the fluid pressures within the chamber and the reference volume are important here. Therefore, the size of the reference volume can be selected independently. In principle, a very small volume can therefore also be considered, so that the device used can be particularly compact.

With a comparatively small volume, it must just be possible to control and/or regulate small amounts of fluid with sufficient precision to adjust the fluid pressure within the reference volume.

In order to perform a non-invasive blood pressure measurement, the method may therefore advantageously provide for the second specific fluid pressure in the reference volume to be increased, for example by inflating the reference volume (for example within said hollow body) with fluid (such as air) (for example by means of the second pump). This is preferably done until the second specific fluid pressure reaches the second initial pressure value.

Aspects of the present disclosure provide a device for non-invasive blood pressure measurement, which comprises a compression unit by means of which a time-varying pressure can be exerted on at least a part of the body of a living being at least during a measuring period, in that a first specific fluid pressure within a chamber of the compression unit can be changed as a function of time at least during the measuring period by means of a first pressure adjustment unit comprised by the device, wherein the device has a differential pressure sensor and an object with an enclosed reference volume and the device is adapted to record with the differential pressure sensor, at different measuring times during the measuring period, at which at least partially different first specific fluid pressures are set, in each case at least one measured value of a differential pressure existing at the respective measuring time between the, in particular current, fluid pressure within the chamber and the, in particular current, fluid pressure within the reference volume, and wherein the device is adapted to adjust a second specific fluid pressure within the reference volume by means of a second pressure adjustment unit comprised by the device, wherein preferably the device is adapted to adjust, by means of the second pressure adjustment unit comprised by the device, the second specific fluid pressure within the reference volume (i) at least during the measuring period and/or time-dependently and/or (ii) in such a way, that at least at at least one, preferably at each, measuring time the, in particular current, fluid pressure within the reference volume is different from at least the first specific fluid pressure set at the respective measuring time and/or the fluid pressure existing in the chamber at the respective measuring time.

All the advantages described in relation to the method according to the first aspect of the disclosure also apply accordingly to the device according to the second aspect of the disclosure. Reference can therefore be made to the previous explanations in this respect.

All features, such as in particular physical embodiments or relative arrangements of individual parts, which have been described in relation to the method according to the first aspect of the disclosure, can advantageously also be provided in the device, individually and in any combination. Furthermore, the device or parts thereof is in each case adapted to perform the obligatory method features and optionally one or more of the optional method features. Reference can therefore be made to the previous explanations in this respect.

In order to carry out the method features, the device may comprise a computing unit, which computing unit is arranged to carry out one or more of the method features, and which computing unit is advantageously operatively connected to one or more other parts of the device so as to be able to influence, monitor, control and/or regulate them respectively as appropriate (for example, the computing unit may: be operatively connected to one or both of the pressure adjustment units and therefore be designed to adjust the first specific fluid pressure in the chamber and/or the second specific fluid pressure in the reference volume as described above; and/or be operationally connected with the differential pressure sensor and therefore be designed to receive and/or evaluate the measured values therefrom as described above).

A third aspect of the present disclosure provides a housing, in particular for a device for non-invasive blood pressure measurement, such as a device according to the second aspect of the invention, is proposed, wherein at least one channel system is integrally formed within the housing, wherein preferably at least one specific section of the channel system has a curved course along a main extension of the specific section.

In this respect, the invention is based on the realization that particularly reliable operation of a device (such as a device for measuring blood pressure) is possible if the fluid lines are fixed and thus better protected against incorrect interconnection or leaks. As the channel system is formed integrally within the housing here, the channel system is protected against manipulation of the fluid lines. As connecting pieces can be dispensed with or at least reduced in number, leaks can also be better prevented. A high level of tightness is particularly advantageous for channel systems that are fluidically connected to and/or form volumes in which certain fluid pressures are set during blood pressure measurements.

It was also recognized that the channel system can be integrally formed particularly well by means of 3D printing and that the support material remaining in the cavities of the channel system after the printing process (which may, for example, have non-consolidated particles) can be removed particularly easily and reliably by making the branches within the channel system at least partially curved (instead of angular, for example).

This is because this curved design allows the support material to be blown out of the duct system comparatively easily by applying compressed air to an opening in the duct system. The same also applies to any cleaning that may be carried out. This is because, in the case of curved sections, the compressed air is, so to speak, fed into the duct section adjoining the curved section. If, on the other hand, branches are angular, for example, the compressed air flow often no longer reaches the duct system or does not reach as far into the duct system, in particular into the downstream part of the duct system behind the branch, so that the duct system cannot be cleared of supporting material and/or cleaned reliably or at all, or only with great effort.

The specific section of the duct system preferably has an inner diameter of 1 mm or more than 1 mm, preferably of 2 mm or more than 2 mm, preferably of 3 mm or more than 3 mm, preferably of 4 mm or more than 4 mm, of 5 mm or more than 5 mm, preferably of 10 mm or more than 10 mm. Alternatively or additionally, the specific section of the channel system preferably has an inner diameter of 50 mm or less than 50 mm, preferably of 30 mm or less than 30 mm, preferably of 20 mm or less than 20 mm, preferably of 15 mm or less than 15 mm, preferably of 10 mm or less than 10 mm, preferably of 7 mm or less than 7 mm, preferably of 5 mm or less than 5 mm, preferably of 3 mm or less than 3 mm.

For example, the specific section of the duct system has an inner diameter of between 1 mm and 10 mm, preferably between 1.5 mm and 8 mm, preferably between 2 mm and 5 mm.

The duct system in question can also be referred to as a first duct system to better distinguish it from other duct systems introduced later.

Alternatively or additionally, it can also be provided that the duct system is formed at least partially, in particular at least the specific section, within a solid housing body.

As a result, the channel system can be particularly well protected, for example against breakage, and the housing can be designed to be stable overall.

Alternatively or additionally, it may also be provided that the housing, the duct system, in particular the specific section, and/or the housing body is at least partially manufactured by means of 3D printing.

As a result, integral structures of the channel system can be realized easily, cost-effectively and with a high degree of precision.

Alternatively or additionally, it can also be provided that the curved course of the specific section has a radius of curvature that is greater than or equal to half of an inner diameter, in particular half of the smallest inner diameter, of the specific section.

It was recognized that a bend formed with these specifications offers particularly good guiding properties for compressed air used to clean the duct system.

For example, the curved course of the specific section can have a radius of curvature that is at least 0.5 times, preferably at least one times, preferably at least 1.5 times, preferably at least 2 times, preferably at least 3 times, preferably at least 4 times, preferably at least 5 times, preferably at least 7 times, preferably at least 10 times the inner diameter of the specific section. Optionally, the radius of curvature is at most 2-fold, preferably at most 3-fold, preferably at most 5-fold, preferably at most 6-fold, preferably at most 7-fold, preferably at most 10-fold, preferably at most 20-fold, of the inner diameter of the specific section. For example, the radius of curvature is between 0.5 times and 20 times, preferably between 1.5 times and 10 times, the inside diameter of the specific section.

For example, the radius of curvature is between 2 mm and 10 mm, preferably between 2.5 mm and 7 mm, preferably between 3 mm and 5 mm.

For example, the smallest or largest radius of curvature of the specific section is meant here.

Alternatively or additionally, it can also be provided that at least one end, preferably both ends, of the specific section is respectively adjoined by a linearly extending sub-section of the duct system. Alternatively or additionally, it can also be provided that at least one end, preferably both ends, of the specific section is or are fluidly connected or connectable to the environment of the housing, in particular in each case via an opening provided on the housing.

The specific section can be connected directly or indirectly to the housing opening. For example, the housing opening can open into the specific section. In this case, the specific section is connected directly to the housing opening, for example. The housing opening can also open into a sub-section of the duct system located between the opening and the specific section. In this case, the specific section is indirectly connected to the housing opening, for example.

Alternatively or additionally, it may also be provided that at least one element, such as a valve, sensor and/or connecting element, is arranged inside the housing and/or on the housing and at least one, in particular fluid pressure-transmitting, fluid-permeable and/or fluid-flowable, connecting piece of the element projects through at least one opening of the housing into the duct system, wherein a tubular and/or plug-shaped sealant is provided, which is arranged in the region of the housing opening between the connecting piece and the housing, at least in certain areas.

The element can be advantageously arranged and/or fixed at least partially on the housing by means of the connecting piece.

In one embodiment, a fluid connection and/or a fluid pressure-transmitting connection between the duct system and at least parts of the element (which are located outside the housing and/or the duct system, for example) can be produced or established by the connecting piece.

By making the sealant tubular or plug-shaped, it can be provided particularly easily between the connecting piece and the housing. For example, the sealant can be detachably or permanently attached to the connecting piece.

The element and the sealant can be designed to be non-destructively detachable or in one piece.

A plug-shaped sealant can, for example, be a sealant that is constructed like a cork. Optionally, the material of the sealant is cork, plastic and/or rubber.

For the purposes of this disclosure, a tubular sealant is preferably understood to mean a sealant which has a length in the main direction of extension of the sealant which is at least one times, preferably at least two times, preferably at least three times, preferably at least five times, preferably at least seven times, preferably at least ten times, preferably at least twenty times, preferably at least thirty times, preferably at least fifty times, preferably at least one hundred times, a diameter, in particular an inner or outer diameter, of the sealant.

For example, the sealant has a length in the main direction of extension of the sealant of between 1 mm and 10 mm, preferably of between 1 mm and 8 mm, preferably of between 2 mm and 8 mm, preferably of between 2 mm and 5 mm.

Alternatively or additionally, it can also be provided that the connecting piece is hollow-cylindrical at least in some sections, the outer diameter of the connecting piece is smaller than the diameter of the housing opening and/or at least one end section of the sealant, preferably the sealant along its entire length, is slipped over the connecting piece, in particular over a part of the connecting piece that is hollow-cylindrical.

This means that the sealant can be provided together with the element, for example, and the sealant can therefore be reliably positioned when the connecting piece is inserted into the housing opening.

Alternatively or additionally, it may also be provided that the sealant at least partially protrudes out of the duct system and/or at least partially protrudes into the duct system and/or comprises a plastic material and/or a rubber material.

As a result, a particularly good seal can be achieved between the connecting piece and the housing along an extended section, in particular along the entire length of the sealant. The rubber material can be silicone or rubber, for example.

Alternatively or additionally, it may also be provided that at least one further channel system is integrally formed within the housing, and preferably at least one specific section of the further channel system has a curved course along a main extension of the specific section, wherein preferably the further channel system is fluidically separated from the at least one other channel system (i.e. in particular the first channel system) or can be separated, in particular by means of a valve which is or can be provided within the housing or on the housing.

For example, a volume with adjustable fluid pressure can be fluidly connected or connectable to each of the at least two channel systems or such a volume can be provided.

In addition, the same advantages as for the at least one channel system, as described above (the first channel system) also apply to the formation of more than one channel system.

The additional duct system can also be referred to as a second duct system to better distinguish it from the first duct system.

For the purposes of this disclosure, the two channel systems are preferably fluidly separated from each other if the two channel systems are still fluidly connected to each other via the bypass of the housing.

The further channel system now described can advantageously have all the features, individually and in any combination, that were described in relation to the first channel system. In particular, the radius of curvature and/or the inner diameter of the specific section of the further duct system can assume the values described above. Unless otherwise apparent from the context, the two channel systems can be designed completely or partially differently, for example with regard to the inside diameter and/or radius of curvature of the specific section.

Alternatively or additionally, for the device according to the second aspect of the disclosure, it may also be provided that the device comprises a housing according to the third aspect of the disclosure.

As a result, all or at least some of the components of the device can be reliably and safely provided within the housing or on the housing and/or brought into operative connection with each other.

For example, the compression unit can be provided outside the housing and can be connected or connectable to the housing via a hose. As a result, the chamber of the compression unit can be very easily and reliably fluidly connected or connectable to the at least one (first/second) channel system and/or the first pressure adjustment unit (in particular via the at least one (first/second) channel system).

Alternatively or additionally, for the device according to the second aspect of the disclosure, it may also be provided that the reference volume is integrally formed within the housing.

This enables a very compact design of the device and reliable operation.

An integral design of the reference volume can be realized, for example, by a volume that is completely enclosed within a housing body, at least in some areas. This enables a high level of tightness. The enclosed volume can then be connected or connectable to one of the channel systems.

For example, the first and/or second channel system forms all or at least part of the reference volume.

Alternatively or additionally, for the device according to the second aspect of the disclosure, it may also be provided that the one channel system (in particular the first or second channel system) of the at least two channel systems is fluidly connected with the reference volume or at least parts of the one channel system form the reference volume and the other channel system (in particular the second or first channel system) of the at least two channel systems is fluidly connected or connectable to the chamber of the compression unit.

For example, a fluid line, such as a hose, can be used to establish a connection between the chamber of the compression unit and the corresponding other channel system. For this purpose, the fluid line can be connected to an opening in the housing that opens into the corresponding other channel system.

Alternatively or additionally, the device according to the second aspect of the disclosure may also provide that the differential pressure sensor is arranged or can be arranged within the housing or on the housing in such a way that the two sides of the differential pressure sensor can be subjected to the pressures existing in the two channel systems and thus a differential pressure between the fluid pressure existing in the reference volume and the fluid pressure existing in the chamber of the compression unit can be measured by means of the differential pressure sensor.

This allows for a particularly compact device.

This means that one side of the differential pressure sensor is advantageously subjected to the fluid pressure in the reference volume and the other side of the differential pressure sensor is subjected to the fluid pressure in the chamber.

Alternatively or additionally, the device according to the second aspect of the disclosure may also provide that (i) the compression unit is provided outside the housing and/or the chamber of the compression unit is fluidly connected or connectable, in particular by means of a fluid line, to at least one of the channel systems (in particular the second channel system), (ii) the first pressure adjustment unit is arranged inside the housing or on the housing, (iii) the second pressure adjustment unit is arranged inside the housing or on the housing, (iv) the differential pressure sensor is arranged inside the housing or on the housing, (v) the chamber of the compression unit is at least partially fluidly connected or connectable to the first pressure adjustment unit via at least one of the channel systems (in particular the second channel system), and/or (vi) the reference volume is formed by at least a part of at least one of the channel systems (in particular the first channel system) and/or the reference volume is fluidly connected or connectable, in particular at least partially via at least one of the channel systems (in particular the first channel system), to the second pressure adjustment unit.

Alternatively or additionally, in the device according to the second aspect of the disclosure, it may also be provided that the valve element is a valve of the first pressure adjustment unit and a fluid connection between a pump of the first pressure adjustment unit and the chamber of the compression unit can be controlled, in particular opened and closed, by means of the valve element.

Alternatively or additionally, for the device according to the second aspect of the disclosure, it may also be provided that the valve element is a valve of the second pressure adjustment unit and a fluid connection between a pump of the second pressure adjustment unit and the reference volume can be controlled, in particular opened and closed, by means of the valve element.

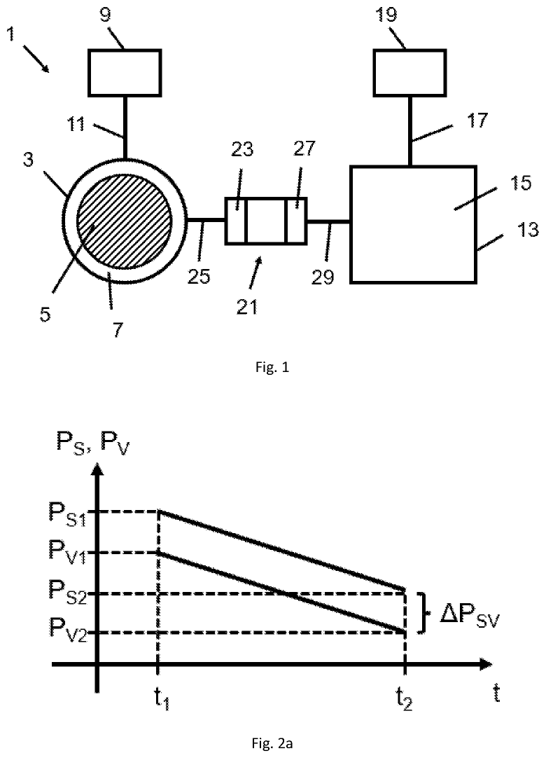

FIG. 1 shows a schematic representation of a device 1 for non-invasive blood pressure measurement.

The device 1 has a compression unit 3 in the form of a blood pressure measuring cuff. The compression unit 3 is placed around a part 5 of the body of a living being, namely around a human upper arm. The upper arm is shown here schematically in a sectional view, with the cut surface hatched.

The compression unit 3 can be used to exert a time-varying pressure on the upper arm 5 by varying a first specific fluid pressure within a chamber 7 of the compression unit 3 as a function of time by means of a first pressure adjustment unit 9 comprised by the device 1. As illustrated in FIG. 1, the chamber 7 circumferentially surrounds the upper arm 5 of the person.

The first pressure adjustment unit 9 is a pump with which fluid, namely air, can be pumped into the chamber 7 and with which the chamber 7 can also be emptied by pumping fluid out of the chamber 7. This allows for the first specific fluid pressure inside the chamber 7 to be adjusted. For this purpose, the pressure adjustment unit 9 is fluidly connected to the chamber 7 via a fluid line 11.

In addition, the device 1 also has a hollow body 13 with an enclosed reference volume 15. In the present case, the hollow body 13 is a cube-shaped body and the reference volume 15 is also cube-shaped. However, a different, for example cylindrical or spherical, design of hollow body and/or reference volume would also be possible.

The reference volume is connected to a second pressure adjustment unit 19 via a fluid line 17. The second pressure adjustment unit 19 is a pump with which fluid, namely air, can be pumped into the reference volume 15 and with which the reference volume 15 can also be emptied by pumping fluid out of it. This allows a second specific fluid pressure to be adjusted within the reference volume 15.

In the present case, the fluid pressure existing in the reference volume 15 is only determined by the respective second specific fluid pressure, i.e. it is identical to it. In contrast, the fluid pressure existing in the chamber 7 is determined by the respective first specific fluid pressure and also by a time-dependent deformation of the cuff 3 and the associated volume change within the chamber 7 due to the force influence that the pulse waves exert on the cuff 3 and thus on the chamber 7 via the blood vessel walls of the human upper arm 5. The time-dependent deformation represents the pulse waves, which can therefore be measured as pressure variations and can be represented, at least mentally, as superimposed on the first specific fluid pressure.

In addition, the device 1 also has a differential pressure sensor 21. The differential pressure sensor 21 has a first side 23, which is fluidly connected to the chamber 7 via a fluid line 25. As a result, the first side 23 of the differential pressure sensor 21 is subjected to the respective fluid pressure within the chamber 7. The differential pressure sensor 21 has a second side 27, which is fluidly connected to the reference volume 15 via a fluid line 29. As a result, the second side 27 of the differential pressure sensor 21 is subjected to the respective fluid pressure within the reference volume 15. In this configuration, the differential pressure value output by the differential pressure sensor 21 corresponds to the value of the fluid pressure within the chamber 7 minus the value of the fluid pressure within the reference volume 15 (or generally “fluid pressure on the first side minus fluid pressure on the second side”).

By means of the first pressure adjustment unit 9, a first specific fluid pressure within the chamber 7 can be adjusted when using the device 1 as described above. And by means of the second pressure adjustment unit 19, a second specific fluid pressure within the reference volume 15 can be adjusted when the device 1 is used as described above.

FIG. 2a shows a diagram of an exemplary course of the first specific fluid pressure Pₛ in the chamber 7 of the compression unit 3 of the device 1, controlled by the first pressure adjustment unit 9, and of an exemplary course of the second specific fluid pressure P_{V} in the reference volume 15, controlled by the second pressure adjustment unit 19.

For example, the first specific fluid pressure P_{S} can be continuously changed, in particular reduced, from a first initial pressure value P_{S1} at time t₁ at a constant rate of change (in mmHg per second) to a first target pressure value P_{S2} at time t₂. For example, the measuring period (t₁ . . . t₂) is 60 seconds. Accordingly, during the same period of time (t₁ . . . t₂), the second specific fluid pressure P_{V} in the reference volume 15 can be continuously changed, in particular reduced, from a second initial pressure value P_{V1} to a second target pressure value P_{V2} at the same rate of change as the first specific fluid pressure P_{S}. At any time, there is an identical pressure difference of ΔP_{SV} between the first specific fluid pressure in the chamber 7 and the second specific fluid pressure in the reference volume 15.

However, apart from the first specific fluid pressure P_{S}, the fluid pressure P_{K} that can actually be measured inside the chamber 7 (against the atmosphere) during the period t₁ to t₂ is still influenced at least by the pulse waves of the living being, which propagate from the heart via the blood vessel walls of the living being, as has already been noted above. The pulse waves exert a variable force on the cuff 3 and thus change the volume of the chamber 7 as a function of time and thus the fluid pressure within the chamber 7.

FIG. 2b shows a diagram of an exemplary and highly schematic representation of the course of the fluid pressure P_{K} in the chamber 7 of the compression unit 3 of the device 1 during the period t₁ to t₂.

The course of the fluid pressure during the period t₁ to t₂ in FIG. 2b can thus be imagined as an additive composition of the course of the first specific fluid pressure (FIG. 2a) and the course of the pressure changes due to the pulse waves. In other words, the pressure changes due to the pulse waves are imprinted on the course of the first specific fluid pressure (FIG. 2a). Conversely, the course of the first specific fluid pressure P_{S} shown in FIG. 2a can also be taken from the course of the actual fluid pressure in FIG. 2b as a “falling baseline” of the pulse waves.

However, if during the period t₁ to t₂ the fluid pressure in the chamber 7 is not measured against the atmosphere, the course of which is shown in FIG. 2b, but is measured against the fluid pressure within the reference volume 15 as the reference pressure, i.e. if the differential pressure ΔP_{KV} is measured, the continuous change in the reference pressure is also taken into account. Since there is always a constant pressure difference of ΔP_{SV} between the first and second specific fluid pressure (FIG. 2a), the course of the differential pressure ΔP_{KV} corresponds to the course of the pulse waves (or the pressure fluctuations caused by them) on a horizontal “baseline”, as schematically illustrated in FIG. 2c.

As can be seen in FIG. 2c, the baseline (shown as a dot-dash line) runs at a pressure value of P_{G}. The value of P_{G} corresponds in particular to the pressure difference ΔP_{SV} between the first and second specific fluid pressure (FIG. 2a), i.e. P_{G}=ΔP_{SV}.

The position of the “baseline” can therefore be shifted by selecting the pressure difference ΔP_{SV}. If the second specific fluid pressure is increased and thus the pressure difference ΔP_{SV} is reduced, the curve in FIG. 2c is shifted downwards. If the first and second specific fluid pressures are identical (and thus the pressure difference is zero), the baseline just coincides with the time axis (t) of the diagram, as shown in FIG. 2d. If the second specific fluid pressure is increased even further, the “baseline” shifts further downwards and with it the course of the pulse waves, as shown in FIG. 2e. For comparison, FIGS. 2d and 2e show the course of the pulse waves in FIG. 2c as a dashed line.

These illustrations clearly show how the value range P_{Min} to P_{Max} of the pressure fluctuations caused by the pulse waves can be shifted by adjusting the second specific fluid pressure, and how the measuring range of a differential pressure sensor can thus be utilized to advantage. For example, the value range can be aligned with P_{Min}>0 so that an A/D converter, that is connected downstream of the differential pressure sensor or that is comprised by the differential pressure sensor, which A/D converter only operates in the positive value range, is optimally fed. Alternatively, the value range can be aligned approximately symmetrically around the zero line (i.e. around the time axis) so that an A/D converter, that is connected downstream of the differential pressure sensor or that is comprised by the differential pressure sensor, which A/D converter operates in the positive and negative value range, is optimally fed.

Back again to the situation as described in relation to FIG. 2c. However, if the lines 25 and 29 are not connected to the first and second sides 23, 27, but are connected to the second and first sides 27, 23 of the differential pressure sensor 21 in reverse, a curve of the pulse wave pressure fluctuations is obtained as shown in FIG. 2f. This corresponds exactly to the curve progression in the diagram in FIG. 2c, but mirrored on a horizontal line.

The following table provides an overview of exemplary parameters of the individual values for non-invasive blood pressure measurement in various living organisms.

| | Living beings | | | |
|---|---|---|---|---|
| Parameters | Human | | Cat | |
| First initial pressure (P_{S1}) | 320 | mmHg | 315 | mmHg |
| Second initial pressure (P_{V1}) | 300 | mmHg | 305 | mmHg |
| First target pressure value (P_{S2}) | 25 | mmHg | 15 | mmHg |
| Second target pressure value (P_{V2}) | 5 | mmHg | 10 | mmHg |

| | | |
|---|---|---|
| Type of pressure change | Continuous with | Continuous with |
| | constant pressure | constant pressure |
| | difference | difference |
| Pressure change rate ((P_{V2} − P_{V1})/ | 3 to 18 mmHg/ | 3 to 18 mmHg/ |
| (t₂ − t₁)) | sec | sec |
| Measuring period (t₂ − t₁) | 12 to 60 | 12 to 60 |
| | seconds | seconds |
| Measured value recording | 500 to 1000/ | 500 to 1000/ |
| | sec | sec |
| Amplitudes of the pulse waves | 0.9 to 2.5 | 0.22 to 0.8 |
| | mmHg | μHg |

FIG. 3 shows a flowchart 100 of a method for non-invasive blood pressure measurement.

In particular with reference to the detailed explanations of device 1 given above, the method advantageously has the following advantageous features.

In 101, during a measuring period (from t₁ to t₂), the compression unit 3 exerts a time-varying pressure on at least the part 5 of the body of a living organism by changing the first specific fluid pressure within the chamber 7 of the compression unit 3 during the measuring period as a function of time by means of the first pressure adjustment unit 9 (for example, from a first initial pressure value to a first target pressure value).

In 103, at least one measured value of the differential pressure existing at the respective measuring time between the (current) fluid pressure within the chamber 7 and the (current) fluid pressure within the reference volume 15 is recorded at different measuring times during the measuring period at which different first specific fluid pressures are set.

The second pressure adjustment unit 19 is used to adjust the second specific fluid pressure within the reference volume 15 in such a way that at least at each measuring time the fluid pressure within the reference volume 15 is different from at least the first specific fluid pressure set at the respective measuring time, namely on the one hand being lower and on the other hand having a constant difference of 25 mmHg (for example from a second initial pressure value to a second target pressure value).

The recorded measured values result in a measurement curve, which represents and/or is and/or makes it possible to determine a course of the pulse waves of the blood pressure of the living being.

In 105 the recorded measured values are evaluated and blood pressure parameters, such as systolic blood pressure and/or diastolic blood pressure of the human person, are determined based on at least one result of the evaluation.

FIG. 4a shows a schematic cross-sectional view of a housing 31.

Within the housing 31, a duct system 33 (which can be referred to in particular as the first duct system) is integrally formed. For this purpose, the duct system 33 is formed within a solid housing body 35. This is particularly simple and reliable, as the housing 31 is manufactured entirely using 3D printing and internal (hollow) structures can therefore be formed in almost any shape.

The duct system 33 has a specific section 37, which has a curved course along a main extension of the specific section 37. The curved course of the specific section 37 has a radius of curvature Rx that is larger than an inner diameter of the specific section 37.

Both ends of the specific section 37 are each connected to a rectilinear sub-section 39 of the duct system 33. In addition, the two ends of the specific section 37 are each fluidly connected or connectable to the surroundings of the housing 31 via rectilinear sections 39 and via openings 41 provided on the housing 31.

Within the housing 31, a further channel system 43 (which may in particular be referred to as a second channel system) is integrally formed. The further duct system 43 also has a specific section 45, which has a curved course along a main extension of the specific section 45. In addition, the two ends of the specific section 45 are each fluidly connected or connectable to the surroundings of the housing 31 via rectilinear sections 47 and via openings 49 provided on the housing 31. The channel system 43 is fluidly separated from the other channel system 33. This means here that the two duct systems 33, 43 are only fluidly connected or connectable to one another via the surroundings of the housing 31.

Due to the curved courses of the specific sections 37, 45, the channel systems 33, 43 can be easily and reliably freed of support material after production by means of 3D printing by applying compressed air to the channel systems 33, 43 via the openings 41 and 49 and thus flushing them. This is because the curved paths of the specific sections 37, 45 guide the compressed air advantageously.

A sensor element in the form of a differential pressure sensor 51 is arranged on the housing 31, although this need not be provided in other embodiments. FIG. 4b shows an enlarged schematic detail representation of a section framed in dashed lines in FIG. 4a. In the detailed view of FIG. 4b, some features of the differential pressure sensor 51 and the adjacent structures of the housing 31 are recognizable.

The differential pressure sensor 51 has two hollow cylindrical connecting pieces 53a, 53b which are permeable to a fluid and through which a fluid can flow (and above all transmit fluid pressure). The first connecting piece 53a projects through the housing opening 41 into the rectilinear section 39 and thus into the duct system 33. The second connecting piece 53b projects through the housing opening 49 into the rectilinear section 47 and thus into the duct system 43. As a result, the two sides of the differential pressure sensor 51 can be subjected to the pressures existing in the two duct systems 33, 43 and the differential pressure existing between these duct systems 33, 43 can thus be determined.

A tubular sealant 55a, 55b is slipped over each of the connecting pieces 53a, 53b. In the arranged state of the differential pressure sensor 51, the respective sealant 55a, 55b is arranged in the region of the respective housing opening 41, 49 between the respective connecting piece 53a, 53b and the housing 31, at least in some areas. The sealant 55a, 55b protrudes at least partially out of the respective duct system 33, 43 and it also protrudes at least partially into the respective duct system 33, 43.

By means of the sealant 55a, 55b, a sealing of the channel systems 33, 43 against the environment of the housing 31 can be achieved particularly advantageously despite manufacturing tolerances (in particular of the housing 31 in 3D printing manufacturing processes).

The housing 31 can be used as a housing for a device for non-invasive blood pressure measurement. For example, this could be a device for non-invasive blood pressure measurement according to of the disclosure, such as the device 1 described above.

FIG. 5 shows a schematic representation of individual components of a device 57 according to the disclosure. The device 57 comprises, inter alia, a housing 31. The housing 31 can be designed similarly or identically to the housing 31 described with reference to FIG. 4a, which is why in FIG. 5 the housing and its parts are provided with the same reference signs as in FIG. 4a.

In the device 57, a reference volume 59 is integrally formed within the housing 31, namely by the channel system 43. The channel system 33 is connected or connectable via a fluid line 61 to a chamber of the compression unit provided outside the housing 31 (neither is shown in FIG. 5). The fluid line 61 can be connectable to the housing 31 (for example by means of a connecting element comprising a connecting piece), whereby a sealant can also be provided which can be designed in a manner similar to the sealant 55a, 55b described with reference to FIG. 4b.

The differential pressure sensor 51 can therefore be used to measure a differential pressure between the fluid pressure in the reference volume 59 and the fluid pressure in the chamber of the compression unit.

A first pressure adjustment unit is arranged on the housing 31, but this is not located in the sectional plane of FIG. 5. The first pressure adjustment unit is fluidly connected to the duct system 33 via a part of the duct system 33 extending perpendicular to the drawing plane of FIG. 5. By means of the first pressure adjustment unit, the fluid pressure within the chamber of the compression unit, which is fluidly connected to the duct system 33 via the fluid line 61, can be adjusted. In addition, a second pressure adjustment unit 63 is arranged on the housing 31, with which the fluid pressure within the duct system 43 and thus within the reference volume 59 can be adjusted.

The second pressure adjustment unit 63 can be connectable to the housing 31 (for example by means of a connecting element comprising a connecting piece), whereby a sealant, which can be designed in a manner similar to the sealant 55a, 55b described with reference to FIG. 4b, can also be provided.

Further components of the device 57 can advantageously be arranged on the housing 31 or within the housing 31, even if these are not described in more detail here.

A housing according to the third aspect of the disclosure (such as the housing 31) can thus advantageously provide structures of channel systems for fluidic connections of different components of a device for non-invasive blood pressure measurement (for example according to the third aspect of the disclosure, such as the device 57). By integrally shaping the channel systems, pressure losses, for example, can be avoided or at least reduced. In addition, the risk of incorrect wiring of the components can be avoided or at least reduced by the predetermined channel systems.

For example, in the device 1 described with reference to FIG. 1, the fluid lines 11, 17, 25, 29 could each be integrally formed in whole or in part as a channel system within a housing according to the third aspect of the disclosure.

While subject matter of the present disclosure has been illustrated and described in detail in the drawings and foregoing description, such illustration and description are to be considered illustrative or exemplary and not restrictive. Any statement made herein characterizing the invention is also to be considered illustrative or exemplary and not restrictive as the invention is defined by the claims. It will be understood that changes and modifications may be made, by those of ordinary skill in the art, within the scope of the following claims, which may include any combination of features from different embodiments described above.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

### LIST OF REFERENCE SYMBOLS

1 Device
3 Compression unit
5 Part of the body of a living being
7 Chamber
9 First pressure adjustment unit
11 Fluid line
13 Hollow body
15 Reference volume
17 Fluid line
19 Second pressure adjustment unit
21 Differential pressure sensor
23 First side of the differential pressure sensor
25 Fluid line
27 Second side of the differential pressure sensor
29 Fluid line
31 Housing
33 Duct system
35 Housing body
37 Specific section
39 Straight section
41 Opening
43 Duct system
45 Specific section
47 Sub-section
49 Opening
51 Differential pressure sensor
53a, 53b Connecting piece
55a, 55b Sealant
57 Device
59 Reference volume
61 Fluid line
63 Pressure adjustment unit
100 Flow chart
101 Exerting a time-varying pressure on a part of the body of a living being
103 Recording measured values of a differential pressure
105 Evaluating the measured values and determining blood parameters of the living being
P_{G} Fluid pressure
P_{K} Fluid pressure
P_{Max} Fluid pressure
P_{Min} Fluid pressure
Pₛ First specific fluid pressure
P_{S1} First specific fluid pressure
P_{S2} First specific fluid pressure
P_{V} Second specific fluid pressure
P_{V1} Second specific fluid pressure
P_{V2} Second specific fluid pressure
ΔP_{SV} Pressure difference
ΔP_{KV} Differential pressure
R_{K} Radius of curvature
t Time axis
t₁, t₂ point in time

1. A method for non-invasive blood pressure measurement, the method comprising:
exerting a time-varying pressure on at least a part of a body of a living being using a compression unit at least during a measuring period,
wherein a first specific fluid pressure within a chamber of the compression unit is changed in a time-dependent manner at least during the measuring period by a first pressure adjustment unit,
wherein at different measuring times during the measuring period, at which at least partially different first specific fluid pressures are set, in each case at least one measured value of a differential pressure that exists at the respective measuring time between the fluid pressure within the chamber and the fluid pressure within a reference volume is recorded, and
wherein a second specific fluid pressure within the reference volume is adjusted by a second pressure adjustment unit.

2. The method according to claim 1, wherein using the second pressure adjustment unit, the second specific fluid pressure within the reference volume is adjusted at least during the measuring period and/or as a function of time.

3. The method according to claim 1, wherein using the second pressure adjustment unit, the second specific fluid pressure within the reference volume is adjusted in such a way, that at least at at least one, preferably at each, measuring time the, in particular current, fluid pressure within the reference volume is different from at least the first specific fluid pressure set at the respective measuring time and/or the fluid pressure existing in the chamber at the respective measuring time.

4. The method according to claim 1, wherein the first specific fluid pressure is set within the chamber, in particular by the first pressure adjustment unit and/or before the measuring period, to a first initial pressure value, which is preferably above a systolic blood pressure of the living being, and preferably the first specific fluid pressure is changed in a time-dependent manner starting from the first initial pressure value, in particular at least during the measuring period, preferably until a first target pressure value, which is preferably below a diastolic blood pressure of the living being, is reached.

5. The method according to claim 1, wherein the first specific fluid pressure is changed continuously or stepwise, in particular at least during the measuring period, by the first pressure adjustment unit.

6. The method according to claim 1, wherein the second specific fluid pressure within the reference volume is set to a second initial pressure value, in particular by the second pressure adjustment unit and/or before the measuring period, and/or is changed as a function of time, in particular by the second pressure adjustment unit, starting from the second initial pressure value and/or at least during the measuring period, preferably until a second target pressure value is reached.

7. The method according to claim 1, wherein the second specific fluid pressure, in particular at least during the measuring period:
is adjusted within the reference volume in such a way that a ratio of the first specific fluid pressure in the chamber and the second specific fluid pressure within the reference volume, in particular at each measuring time point, has a value (a) of between 0.5 and less than 1.0, in particular of between 0.6 and less than 1.0, in particular of between 0.7 and less than 1.0, in particular of between 0.9 and less than 1.0, and/or (b) of between more than 1.0 and 1.5, in particular of between more than 1.0 and 1.4, in particular of between more than 1.0 and 1.3, in particular of between more than 1.0 and 1.1,
is changed within the reference volume at least temporarily simultaneously with the first specific fluid pressure within the chamber,
is changed within the reference volume in phase with the first specific fluid pressure within the chamber,
has within the reference volume a pressure difference in terms of magnitude with respect to the first specific fluid pressure within the chamber of at least one times, preferably at least two times, preferably at least three times, preferably at least four times, preferably at least five times, the maximum pressure change caused by pulse waves during the measuring period,
and/or
is changed within the reference volume at a rate of change identical to that of the first specific fluid pressure within the chamber.

8. The method according to o claim 1, wherein, in particular at least at the individual measuring times, the pressure difference between the first specific fluid pressure within the chamber and the second specific fluid pressure within the reference volume is set (i) to a constant value and/or (ii) to a value of (a) 1 mmHg or more than 1 mmHg, (b) 600 mmHg or less than 600 mmHg and/or (c) between 1 mmHg and 600, preferably between 1 mmHg and 100 mmHg, preferably between 10 mmHg and 50 mmHg, such as 1 mmHg, 10 mmHg, 25 mmHg, 30 mmHg or 50 mmHg.

9. The method according to claim 1, wherein the, in particular current, fluid pressure within the reference volume is not influenced by the living being or parts thereof, in particular not by the blood pressure of the living being and/or by pulse waves propagating via the blood vessels of the living being.

10. The method according to claim 1, wherein the first specific fluid pressure in the chamber and the second specific fluid pressure in the reference volume are controlled and/or adjusted, in particular at least during the measuring period and/or at least for each measurement, in such a way that, in particular at least during the measuring period and/or at least for each measurement, the first specific fluid pressure is, preferably at any time, at a greater or smaller value than the second specific fluid pressure.

11. The method according to claim 1, wherein the recorded measured values result in a measurement curve which represents and/or is and/or makes it possible to determine a course of pulse waves of the blood pressure of the living being.

12. The method according to claim 1, wherein the differential pressure between the, in particular current, fluid pressure in the chamber and the, in particular current, fluid pressure in the reference volume is measured by a differential pressure sensor, wherein preferably a first side of the differential pressure sensor is fluidly connected to the chamber and a second side of the differential pressure sensor is fluidly connected to the reference volume, and in particular the differential pressure sensor supplies positive pressure values, if the, in particular current, fluid pressure in the chamber is greater than the, in particular current, fluid pressure in the reference volume, and the differential pressure sensor supplies negative pressure values if the, in particular current, fluid pressure in the chamber is less than the, in particular current, fluid pressure in the reference volume.

13. The method according to claim 1, wherein the compression unit comprises or is a blood pressure measuring cuff, wherein preferably the part of the body of the living being can be compressed to varying degrees by the cuff, and/or wherein the chamber is a chamber of the cuff, which preferably can be filled with a fluid and emptied.

14. A device for non-invasive blood pressure measurement, the device comprising:
a compression unit comprising a chamber and configured to exert a time-varying pressure on at least a part of a body of a living being at least during a measuring period;
a first pressure adjuster comprising a first pump configured to change a first specific fluid pressure within the chamber of the compression unit as a function of time at least during the measuring period; and
a differential pressure sensor and an object with an enclosed reference volume,
wherein the device is configured to record with the differential pressure sensor at different measuring times during the measuring period, at which at least partially different first specific fluid pressures are set, in each case at least one measured value of a differential pressure existing at the respective measuring time between the fluid pressure within the chamber and the fluid pressure within the reference volume, and
wherein the device further comprises a second pressure adjuster comprising a second pump and configured to adjust a second specific fluid pressure within the reference volume.

15-25. (canceled)

26. The device according to claim 14, the device further comprising a housing, wherein at least one channel system is integrally formed within the housing, wherein the reference volume is integrally formed within the housing.

27. The device according to claim 26, wherein the one channel system of the at least two channel systems is fluidly connected to the reference volume or at least parts of the one channel system form the reference volume and the other channel system of the at least two channel systems is fluidly connected or connectable to the chamber of the compression unit.

28. The device according to claim 26, wherein the differential pressure sensor is arranged or is configured to be arranged within the housing or on the housing in such a way that the two sides of the differential pressure sensor can be subjected to the pressures existing in the two channel systems and thus a differential pressure between the fluid pressure existing in the reference volume and the fluid pressure existing in the chamber of the compression unit can be measured by the differential pressure sensor.

29-31. (canceled)
